(19) United States
(12) Patent Application Publication
(10) Pub. No.: US 2002/0172983 A1
(43) Pub. Date: Nov. 21, 2002

(54) Diagnosis of pathologies of mononucleated blood cells

(76) Inventors: Denis Barritault, Paris (FR); Ammar Achour, Creteil (FR); Jose Courty, Villecresnes (FR); Francoise Baudoin, Boulogne-Billancourt (FR)

Correspondence Address: SCHNADER HARRISON SEGAL & LEWIS, LLP, 1600 MARKET STREET, SUITE 3600, PHILADELPHIA, PA, 19103

(21) Appl. No.: 10/116,076

(22) Filed: Apr. 4, 2002

Related U.S. Application Data
(63) Continuation of application No. PCT/FR00/02788, filed on Oct. 6, 2000.

(30) Foreign Application Priority Data
Oct. 12, 1999 (FR) 99/12715

(51) Int. Cl. G01N 33/567

(52) U.S. Cl. 435/7.21

(57) ABSTRACT

A process for detecting B lymphocytes including contacting a blood sample from a subject, or a fraction of the blood sample, with HARP polypeptide, a fragment or a derivative thereof, and detecting binding to the surface of B lymphocytes by polypeptide HARP, a fragment or a derivative thereof.

### RELATED APPLICATION

This is a continuation of International Application No. PCT/FR00/02788, with an international filing date of Oct. 6, 2000, which is based on French Patent Application No. FR 99/12715, filed Oct. 12, 1999.

### FIELD OF THE INVENTION

This invention concerns a process using the HARP growth factor for detecting certain mononucleated cells in blood and is, thus, useful for diagnosing proliferative pathologies of this type of cell such as, for example, chronic lymphoid leukemias.

### BACKGROUND

The lymphoproliferative syndromes group together diverse lymphoid pathologies characterized by an augmentation in the number of circulating lymphocytes or by morphological anomalies of the pathological lymphocytes. The possibility of specific therapies for certain types of lymphoproliferative syndromes requires a precise diagnosis which is not always possible on the basis of clinical examination or cytological study. At present, there are no specific immunological markers of the different lymphoproliferative syndromes. Diagnosis remains very often difficult despite efforts during recent years to develop a classification system that can be used for the cases. The diagnostic endeavor is based on analyzing a set of markers, certain of which have proven pertinent in discriminating between the two large classes of lymphoproliferative syndromes which are the chronic lymphoid leukemias (CLL) and the malignant non-Hodgkin's lymphomas (NHL). These markers are CD5, CD23, FMC7, CD22 and the surface immunoglobulins.

The immunological phenotype characteristics of the CLL diseases, many of which are indolent (CD5+, CD23+, FMC7−, weak CD20, weak CD22, weak surface immunoglobulins (slg), distinguish them from another much more aggressive B CD5+ lymphoproliferative syndrome, NHL of the mantle (CD5+, CD23−, FMC7+, strong CD20, strong sIg).

Tricholeukocyte leukemia, in turn, has the following typical phenotype: CD5−, strong CD11c, CD25+, CD103+, which differentiates it from the other B CD5-lymphoid hemopathies as well as from splenic NHL with villous lymphocytes, which is morphologically very close to it. Moreover, in patients who are CLL carriers, a barely typical immunophenotype can be associated with a trisomy 12 or a morphology of the mixed cells [1]. These markers have finally made it possible to establish scores, sums of points attributed as a function of their expression, in perpetual observation [2].

Despite this progress and the confrontation with the various clinical, cytological, histological and cytogenetic aspects, many cases still remain contentious. Numerous research projects are in progress seeking to discover new molecules which will make it possible to better discriminate the CLL diseases, which are the most frequently occurring leukemias in the Western world, from the other more aggressive lymphoproliferative syndromes.

Known in the prior state of the art are numerous angiogenic growth factors such as the factors HARP, MK, FGF-1, FGF-2, VEGF, HIV1-tat, HIV2-tat, HGF, HB-EGF and angiogenin.

HARP (Heparin Affin Regulatory Peptide), also called PTN (pleiotrophin) or HB-GAM (heparin binding-growth associated molecule), constitutes with MK (midkine) a family of structurally related growth/differentiation factors that bind to heparin, presenting 50% homology in amino acids [3, 4].

The HARP growth factor is a polypeptide of 168 amino acids containing a N-terminal hydrophobic motif of 32 amino acids corresponding to a signal peptide. In its mature form, HARP is a secreted protein of 136 amino acids (in its short form) or 139 amino acids (in its long form), the apparent molecular weight of which is 18 kDa, determined in SDS-PAGE under reducing conditions.

HARP was initially isolated from neonatal rat brains as a molecule inducing in vitro neurite growth [5], suggesting that this polypeptide is involved in the maturation of neuronal cells [6].

Subsequent studies showed that this polypeptide was also present in non-neuronal tissues, such as the heart [7], the uterus [8], cartilage [9] and bone extracts [10], demonstrating that the function of HARP is not limited to a promotional action on neurite growth as previously reported [5].

HARP is capable of stimulating the growth of fibroblastic, epithelial and endothelial cells in vitro [8, 9]. This mitogenic action has since been confirmed by the use of recombinant proteins produced from eukaryote expression systems [9, 10, 11]. HARP also induces in vitro formation of pseudocapillaries [10]. In vivo, in different tissue models, localization of HARP is especially associated with endothelial cells of blood capillaries [11]. The data concerning HARP available at present suggest that this polypeptide plays a role in complex mechanisms involved in angiogenesis and in tumor neoangiogenesis. Extensive research has been performed regarding this aspect to determine the involvement of HARP in tumoral progression, particularly, in the hormone-dependent tumors such as the breast and the prostate.

Studies pertaining to the biological properties of HARP have been performed by numerous laboratories [4] and, despite much debated results, it would appear that HARP, like MK, is involved in the control of cellular proliferation [4, 9, 12].

Moreover, it has been demonstrated that purified human recombinant HARP (hrHARP) protein is mitogenic for endothelial cells [9, 10] and exerts in vitro an angiogenic action [10].

HARP is thus known and used for its angiogenic and neurotrophic properties.

### SUMMARY OF THE INVENTION

This invention relates to a process for detecting B lymphocytes including contacting a blood sample from a subject, or a fraction of the blood sample, with HARP polypeptide, a fragment or a derivative thereof, and detecting binding to the surface of B lymphocytes by polypeptide HARP, a fragment or a derivative thereof.

This invention also relates to a process for diagnosing proliferative pathologies of mononucleated cells of blood, including detecting B lymphocytes in a patient's blood sample by contacting a blood sample from a subject, or a fraction of the blood sample, with HARP polypeptide, a fragment or a derivative thereof, detecting binding to the surface of B lymphocytes by polypeptide HARP, a fragment or a derivative thereof, measuring the binding rate of HARP on the B lymphocytes, and comparing the resulting rate to a control value obtained in a healthy subject to associate augmentation of binding of HARP with augmentation in the B lymphocytes and with a proliferative pathology of the mononucleated cells of the blood.

### BRIEF DESCRIPTION OF THE DRAWINGS

Other advantages and characteristics of the invention will become manifest from the examples below in which reference will be made to the attached drawings in which:

FIG. 1 shows the binding of HARP on PBMCs obtained from healthy subjects or from subjects with chronic lymphoid leukemia. The solid bars correspond to the cells from healthy blood donors. The clear bars correspond to the cells obtained from patients suffering from chronic lymphoid leukemia.

FIG. 2 shows histograms of conditioned biparametric fluorescence obtained with a flow cytometer on the acquisition window of the lymphocytes of normal subjects, obtained from a cytogram representing the cell size in relation to the cell structure wherein:

a=CD19-PE double labeling on the ordinate versus HARP-FITC on the abscissa in the absence of hrHARP;

b=CD2-PC5 double labeling on the ordinate versus HARP-FITC on the abscissa in the absence of hrHARP;

c=CD19-PE double labeling on the ordinate versus HARP-FITC on the abscissa in the presence of hrHARP;

d=CD2-PC5 double labeling on the ordinate versus HARP-FITC on the abscissa in the presence of hrHARP.

FIGS. 3a and 3b show histograms of conditioned fluorescence on malignant lymphoid cells (CLL): in the absence of human recombinant HARP (hrHARP) (FIG. 3a) and in the presence of hrHARP (FIG. 3b).

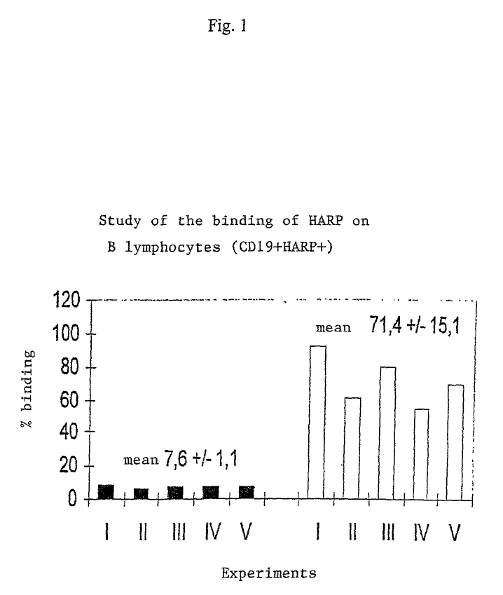

### DETAILED DESCRIPTION

We observed the presence of mRNA corresponding to the HARP protein in cells of blood vessels, both in endothelial cells and smooth muscle cells, but also in smooth muscle cells of human mammary glands [11].

The data reporting that HARP is an angiogenic growth factor and that it is synthesized and localized in the vascular endothelial cells led us to investigate the potential function of HARP on blood cells. We, thus, investigated to see if HARP can bind to these blood cells, especially freshly isolated human mononucleated cells of peripheral blood (PBMC). We demonstrated that HARP binds specifically on the B cells characterized by the presence of the marker CD19. The presence of HARP binding sites on circulating cells has never been described until now.

Our knowledge of the HARP receptors is very limited at present. The presence of very strong affinity HARP binding sites (Kd=600 pM) in NIH 3T3 cells has already been reported [13]. These HARP binding sites have also been found in various cell types, including rat kidney cells, human mammary adenocarcinoma cells, human epidermal carcinoma cells, human hepatocarcinoma cells, mouse neuroblasts and pheochromocytoma cells.

We recently showed that the HARP growth factor, a molecule known to be an angiogenic and neurogenic factor, stimulates production of cytokines of inflammation (IL-1, IL-6, IL-8, IFN-gamma and TNF-alpha) and augments incorporation of tritiated thymidine by mononucleated cells of circulating blood of normal subjects after 7 days of culture. The intensity of this effect is stronger if the cells are quiescent at the beginning. As a result of these findings, we became interested in the relationships of this molecule with the hemopoietic cells and, more especially, with the lymphocytes of normal subjects and of patients who are carriers of malignant hemopathies.

We have now discovered that HARP is capable of binding specifically on the B lymphocyte cells and can, thus, enable the diagnosis of pathologies in which these cells are implicated, and during which the number of B lymphocytes is considerably augmented, as is the case for chronic lymphoid leukemias.

The invention, thus, pertains to a process for detection of B lymphocytes comprising the following steps:

bringing into contact a blood sample from a subject, or a fraction of this blood sample, with the HARP polypeptide, a fragment or a derivative of HARP;

detection of the binding to the surface of the B lymphocytes by the polypeptide HARP, a fragment or a derivative of HARP by any suitable means.

A blood sample can be collected from healthy subjects or from subjects with proliferative pathologies of the mononucleated cells of the blood. This sample is brought into contact with the HARP polypeptide under conditions that promote the peptide binding reaction. In the detection process according to the invention, the blood sample can be brought into contact with:

the HARP polypeptide, i.e., a protein whose amino acid sequence corresponds to the amino acid sequence given in the literature [3, 5, 8];

a fragment of HARP, either a protein or a peptide capable of binding on B lymphocytes and whose amino acid sequence corresponds to a part of the amino acid sequence presented as an attachment as number SEQ ID No. 1;

a derivative of HARP, i.e., a peptide or a polypeptide capable of binding on B lymphocytes and whose amino acid sequence is close to the amino acid sequence identified in the attachment as number SEQ ID No. 1. The term “derivative of HARP” is also understood to mean a protein capable of binding on B lymphocytes and comprising a part or the totality of the amino acid sequence corresponding to the amino acid sequence represented as an attachment as number SEQ ID No. 1 associated with another element of a protein or non-protein nature. This element advantageously enables detection of the binding of the HARP derivative on B lymphocytes. This element can be, for example, a radioactive element or an amino acid sequence coding for an enzyme whose chromogen substrate can also be added to the reaction medium. Another possibility is, for example, covalent association of biotin with HARP, enabling visualization of HARP-B lymphocytes interaction by addition of streptavidin.

The process according to the invention can be applied by bringing the HARP polypeptide, a fragment or a derivative of HARP into contact with a sample of whole blood.

According to another aspect of implementation of the process, a step during which the mononucleated blood cells are separated from the whole blood sample is performed prior to bringing the cells into contact with the HARP polypeptide, a fragment or a derivative of HARP.

The process according to the invention can be implemented in a manner such that binding to the surface of mononucleated blood cells by the HARP polypeptide, a fragment or a derivative of HARP is detected directly.

Direct detection of the binding of HARP, a fragment or a derivative of HARP can be performed by using, for example, a radioactively labeled HARP polypeptide. It is also possible to use a HARP polypeptide coupled to an enzyme or biotin and, in this case, the binding of HARP to B lymphocytes would be detected by adding a corresponding chromogen substrate or streptavidin. It is also possible to react a blood sample with the HARP polypeptide, a fragment or a derivative of HARP, then with a fluorescent antibody recognizing specifically the HARP polypeptide, the fragment or the derivative of HARP employed in the reaction. The binding of the anti-HARP antibodies on the cells can then be detected by flow cytofluorimetry.

The process according to the invention can also be implemented in a manner such that binding to the surface of mononucleated blood cells by the HARP polypeptide, a fragment or a derivative of HARP is detected indirectly.

In one of the implementations of the process according to the invention for the indirect detection of the binding of HARP on B lymphocytes, a blood sample is brought into contact with the HARP polypeptide, a fragment or a derivative of HARP. Then an antibody is added which binds specifically to the HARP polypeptide, a fragment or a derivative of HARP. Finally, a fluorescent antibody binding specifically to the first antibody is added. Binding of the anti-antibody antibodies on the cells can then be detected by flow cytofluorimetry.

The invention has as an advantage in particular a process for diagnosing proliferative pathologies of the mononucleated cells of the blood. This process is characterized in that the B lymphocytes are detected in a patient's blood sample. Then, the binding rate of HARP on the B lymphocytes is measured and this rate is compared to a control value obtained in a healthy subject, in a manner such as to associate an augmentation of binding of HARP with an augmentation in the B lymphocytes and with a proliferative pathology of the mononucleated cells of the blood.

According to one of the possible applications of the invention, the HARP factor is advantageously used for diagnosing proliferative pathologies of mononucleated cells of the blood, such as chronic lymphoid leukemias.

Another aspect of the invention concerns an element of mononucleated cells of blood enabling the binding of the HARP factor on said cells. This element can especially be a receptor that binds the HARP factor exclusively or non-exclusively.

### EXAMPLE 1

### Use of HARP as a Marker of the Lymphocytes of Patients Presenting with a Chronic Lymphoid Leukemia

Mononucleated cells were isolated from peripheral blood of normal subjects (blood donors) or subjects who were carriers of various hemopathies: 30 subjects stricken with CLL, 6 with NHL B, 4 with Sezary's disease, 2 with acute myeloid acute leukemia (AML2 and AML3) and 1 with chronic myeloid leukemia (CML). The blood was collected on a Vacutainer tube containing EDTA. The mononucleated cells were separated by ficoll gradient, counted and adjusted to 10⁶ cells per ml. The HARP growth factor employed was human recombinant HARP (hrHARP) protein of 139 amino acids, obtained from the CRRET laboratory (ESA CNRS 7053) at Creteil Universite, Paris 12 (France). The anti-HARP antibody (human anti-HARP goat immunoglobulin) obtained from the company R&D Systems, Minneapolis (Minnesota, USA) was used in a final dilution of 1/250. A goat anti-IgG antibody coupled to FITC (obtained from Caltag, Burlingame, Calif., USA) at a dilution of 1/50 was used as a secondary antibody. As a negative control, the anti-HARP was replaced by goat serum (obtained from the Jackson Company, West Grove, Pa., USA).

Other antibodies were employed for identifying the cell populations: CD19-PE, CD2-PC5, CD10-FITC, CD45Ra-FITC, CD45Ro-FITC, CD4-PE, CD8-ECD, CD3-FITC, CD16/56-PE, CD25-FITC (all of these antibodies were obtained from the Beckman Coulter-Immunotech Company, Hialeah, Fla., USA).

The cells were separated by ficoll gradient then washed in PBS-0.1% BSA to measure the endogenous content of HARP growth factor in the circulating cells. These cells were then incubated with the primary anti-HARP antibody for 30 minutes at room temperature, washed in PBS-0.1% BSA and returned to incubation with the secondary antibody for 30 minutes. After washing and fixation in 1% formaldehyde, the cells were analyzed using the XL cytofluorimeter (Beckman-Coulter, Hialeah, Fla., US).

The mononucleated cells were incubated with human recombinant HARP (hrHARP) at a concentration of 1 μg/ml for 1 h at room temperature, then washed, prior to being labeled according to the preceding technique to investigate the HARP binding site(s) of the cells. The effect of hrHARP on normal mononucleated cells after 5 days of culture was studied in a humid atmosphere at 37° C. enriched with 5% CO₂ and in the presence of 1 μg/ml of hrHARP. After being washed, the cells were labeled with anti CD19, CD2, CD4, CD8 monoclonal antibodies as well as with the anti-HARP. The cytofluorimetric reading was performed using a 488-nm laser beam. A window of acquisition was drawn around the lymphocytes on a biparametric histogram representing the cell size according to a linear mode in relation to the logarithm of the granulometry. Histograms representing the fluorescent intensity in logarithmic mode in relation to the number of cells was thereby obtained from this selected cell population. The results were acquired as percentages of cells.

The mononucleated cells were analyzed positive for the antibody concerned. Biparametric histogram representing the expression of fluorescence of two different antigens labeled with different fluorochromes enabled analysis of the CD19/HARP double labelings.

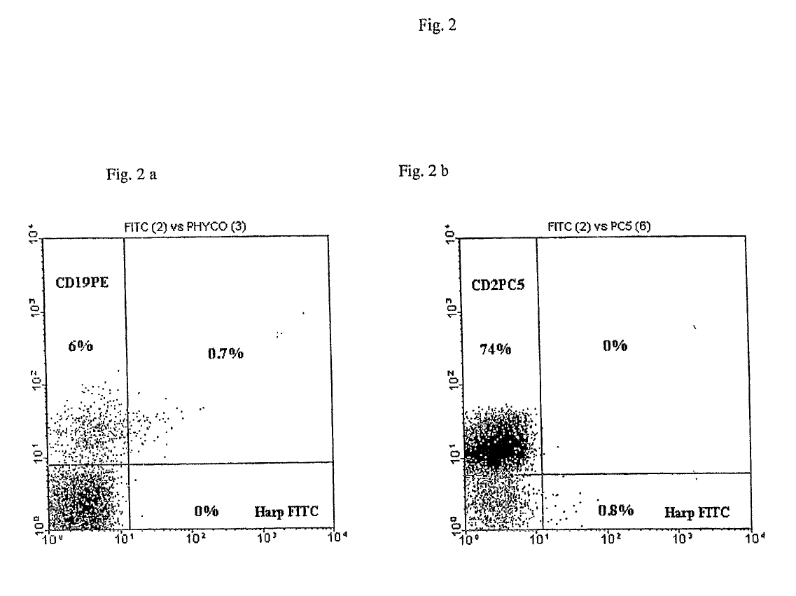

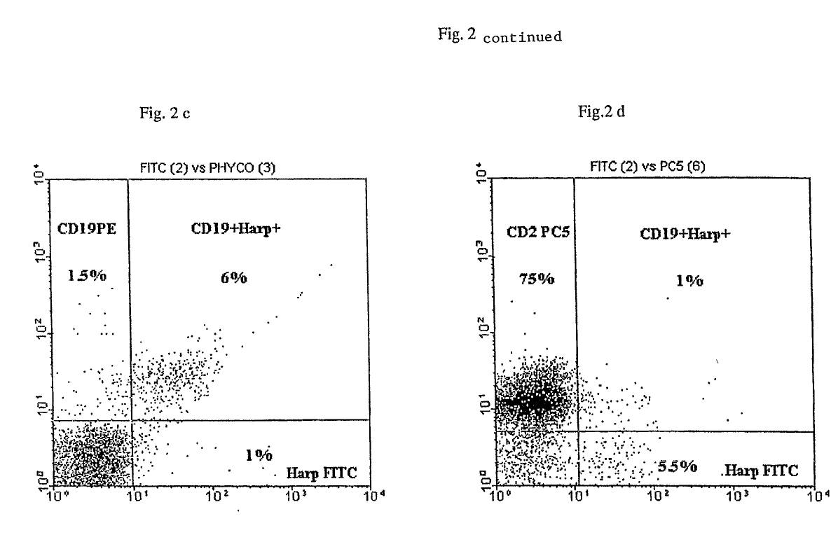

The results for the investigation on the presence of endogenous HARP were negative and no direct expression of HARP was detected on the lymphocytes of the 10 normal subjected tested, nor on the malignant lymphoid and myeloid cells tested, after separation of the mononucleated cells by ficoll gradient. Investigation of cellular HARP binding sites revealed HARP binding on the B lymphocytes of normal subjects. It should be noted that for 2 subjects out of the 10 subjects analyzed, the CD2 positive cells were also capable of binding hrHARP without it being possible to make a distinction between the T and NK cells. The conditioned biparametric fluorescence histograms on the window of acquisition of the lymphocytes of normal subjects obtained from a cytogram representing cell size in relation to cell structure are shown in FIG. 2. Double labeling with CD19-PE versus HARP-FITC (FIG. 2a) and CD2-PC5 versus HARP-FITC (FIG. 2b) in the absence of hrHARP, on the one hand, and the same labelings (FIGS. 2c and 2d) in the presence of hrHARP, on the other hand, show binding of HARP associated with CD19 in the healthy subjects.

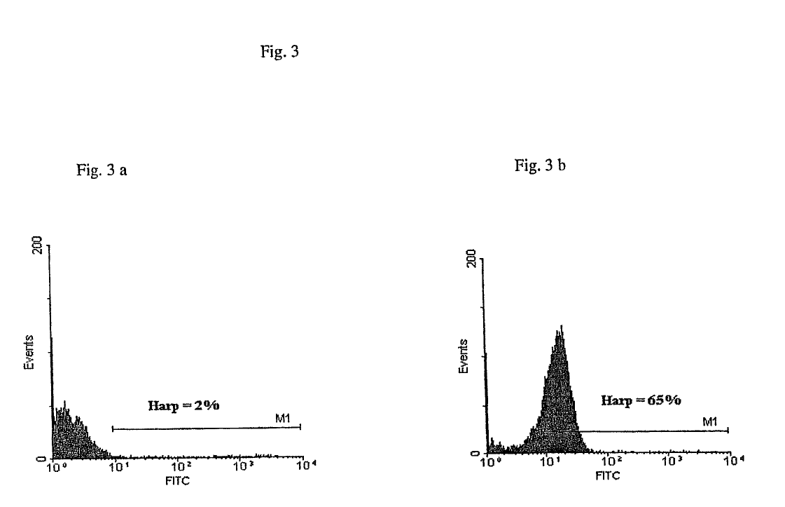

Binding of HARP on lymphocytes was observed in patients with certain CLL and NHL diseases. This was demonstrated by triple labelings with a B lymphoid cell marker, CD19, a pan T marker, CD2, and HARP. 100% of the B lymphoid cells bound HARP whereas the CD2 positive cells did not bind it. Furthermore, it appears that the cells expressing CD10 can not bind HARP under the conditions of our study. These results are shown in FIG. 3, which are histograms of conditioned fluorescence on malignant lymphoid cells: in the absence of hrHARP (FIG. 3a) and in the presence of hrHARP (FIG. 3b). Furthermore, the pathological myeloid cells from the two patients with AML and the one with CML as well as the phenotype T lymphomatous cells from Sezary disease did not exhibit binding of hrHARP.

### BIBLIOGRAPHIC REFERENCES

1) DiGiuseppe J A, Borowitz M J, Clinical utility of flow cytometry in the chronic lymphoid leukemias. Semin Oncol 1998, 25(1):6-10).

2) Matutes E. et al. The immunological profile of B-cell disorders and proposal of a scoring system for the diagnosis of CLL. Leukemia 1994, 8(10):1640-5).

3) Bohlen P and Kovesdi I. 1991. HBNF and MK, members of a novel gene family of heparin-binding proteins with potential roles in embryogenesis and brain function. Prog. Growth Factor Res. 3:143

4) Laaroubi K, Vacherot F, Delbe J, Caruelle D, Barritault D and Courty J. 1995. Biochemical and mitogenic properties of the heparin-binding growth factor HARP. Prog. Growth Factor Res. 6:25).

5) Rauvala H. 1989. An 18-kd heparin-binding protein of developing brain that is distinct from fibroblast growth factors. EMBO J. 8:2933.

6) Merenmies J and Rauvala H. 1990. Molecular cloning of the 18-kDa growth associated protein of developing brain. J. Biol. Chem. 265:16721.

7) Hampton B S, Marshak D R and Burgess W H. 1992. Structural and functional characterization of full-length heparin-binding growth associated molecule Mol. Biol. Cell. 3:85).

8) Milner P G, Li Y S, Hoffman R M, Kodner C M, Siegel N R and Deuel T F. 1989. A novel 17 kD heparin-binding growth factor (HBGF-8) in bovine uterus: purification and N terminal amino acid sequence. Biochem. Biophys. Res. Commun. 165:1096.

9) Fang W, Hartmann N, Chow D T, Riegel A T and Wellstein A. 1992. Pleiotrophin stimulates fibroblasts and endothelial and epithelial cells and is expressed in human cancer. J. Biol. Chem. 267:25889).

10) Laaroubi K, Delbe J, Vacherot F, Desgranges P, Tardieu M, Jaye M, Barritault D and Courty J. 1994. Mitogenic and in vitro angiogenic activity of human recombinant heparin affin regulatory peptide. Growth Factors 10:89.

11) Ledoux D, Caruelle D, Sabourin C, Liu J, Crepin M, Barritault D and Courty J. 1997. Cellular distribution of the angiogenic factor heparin affin regulatory peptide (HARP) mRNA and protein in the human mammary gland. J. Histochem. Cytochem. 45:1.

12) Wellstein A et al. 1992. A heparin-binding growth factor secreted from breast cancer cells homologous to a developmentally regulated cytokine. J. Biol. Chem. 267:2582.

13) Kuo MD, Huang SS and Huang JS. 1992. Characterization of heparin-binding growth-associated factor receptor on NIH 3T3 cells. Biochem. Biophys. Res. Commun. 182:188).

1. A process for detecting B lymphocytes comprising:
contacting a blood sample from a subject, or a fraction of the blood sample, with HARP polypeptide, a fragment or a derivative thereof; and
detecting binding to the surface of B lymphocytes by polypeptide HARP, a fragment or a derivative thereof.

2. The process according to claim 1, wherein the HARP polypeptide, a fragment or a derivative thereof is contacted with a sample of whole blood.

3. The process according to claim 1, further comprising separating mononucleated blood cells from other components of the blood sample prior to contacting the cells with the HARP polypeptide, a fragment or a derivative thereof.

4. The process according to claim 1, wherein binding to the surface of the mononucleated blood cells by the HARP polypeptide, a fragment or a derivative thereof is detected directly.

5. The process according to claim 2, wherein binding to the surface of the mononucleated blood cells by the HARP polypeptide, a fragment or a derivative thereof is detected directly.

6. The process according to claim 3, wherein binding to the surface of the mononucleated blood cells by the HARP polypeptide, a fragment or a derivative thereof is detected directly.

7. The process according to claim 1, wherein binding to the surface of the mononucleated blood cells by the HARP polypeptide, a fragment or a derivative thereof is detected indirectly.

8. The process according to claim 2, wherein binding to the surface of the mononucleated blood cells by the HARP polypeptide, a fragment or a derivative thereof is detected indirectly.

9. The process according to claim 3, wherein binding to the surface of the mononucleated blood cells by the HARP polypeptide, a fragment or a derivative thereof is detected indirectly.

10. A process for diagnosing proliferative pathologies of mononucleated cells of blood, comprising:
detecting B lymphocytes in a target subject's blood sample by contacting the blood sample or a fraction of the blood sample, with HARP polypeptide, a fragment or a derivative thereof;
detecting binding to the surface of B lymphocytes by polypeptide HARP, a fragment or a derivative thereof;
measuring the binding rate of HARP on the B lymphocytes; and
comparing the resulting rate to a control value obtained in a healthy subject to associate augmentation of binding of HARP with augmentation in the B lymphocytes and with a proliferative pathology of the mononucleated cells.

11. The process according to claim 10, wherein the blood sample is a sample of whole blood.

12. The process according to claim 10, further comprising separating mononucleated blood cells from other components of the blood sample prior to contacting the cells with the HARP polypeptide, a fragment or a derivative thereof.

13. The process according to claim 10, wherein binding to the surface of the mononucleated blood cells by the HARP polypeptide, a fragment or a derivative thereof is detected directly.

14. The process according to claim 11, wherein binding to the surface of the mononucleated blood cells by the HARP polypeptide, a fragment or a derivative thereof is detected directly.

15. The process according to claim 12, wherein binding to the surface of the mononucleated blood cells by the HARP polypeptide, a fragment or a derivative thereof is detected directly.

16. The process according to claim 10, wherein binding to the surface of the mononucleated blood cells by the HARP polypeptide, a fragment or a derivative thereof is detected indirectly.

17. The process according to claim 11, wherein binding to the surface of the mononucleated blood cells by the HARP polypeptide, a fragment or a derivative thereof is detected indirectly.

18. The process according to claim 12, wherein binding to the surface of the mononucleated blood cells by the HARP polypeptide, a fragment or a derivative thereof is detected indirectly.
